(19) United States
(12) Patent Application Publication
(10) Pub. No.: US 2004/0259932 A1
(43) Pub. Date: Dec. 23, 2004

(54) Method and compound to reduce the incidence of diabetes in a subject with chronic heart failure

(76) Inventors: Martial G. Bourassa, Iles Laval (CA); Jean-Claude Tardif, Laval (CA); Anique Ducharme, Montreal (CA)

Correspondence Address: Ms. Diane Dunn McKay, MATHEWS, COLLINS, SHEPHERD & McKAY, P.A, Suite 306, 100 Thanet Circle, Princeton, NJ, 08540-3674, US

(21) Appl. No.: 10/796,936

(22) Filed: Mar. 10, 2004

Related U.S. Application Data
(60) Provisional application No. 60/453,767, filed on Mar. 11, 2003.

(51) Int. Cl. A61K 31/401

(52) U.S. Cl. 514/423

(57) ABSTRACT

The present invention relates to the effect of angiotensin-converting enzyme (ACE) inhibitors on the prevention of diabetes in a subject with left ventricular dysfunction. A retrospective study was conducted to assess the effect of the ACE inhibitor enalapril on the incidence of diabetes in a group of patients from the Montreal Heart Institute enrolled in the Studies of Left Ventricular Dysfunction (SOLVD). Clinical charts were evaluated for fasting plasma glucose (FPG) levels by blinded reviewers. A diagnosis of diabetes was made when a FPG≧126 mg/dL (7 mmol/L) was found at 2 visits (follow-up, 2.9±1.0 years). Of the 291 non-diabetic patients enrolled in the SOLVD study, 153 of these were on enalapril and 138 were on placebo. Baseline characteristics were similar in the 2 groups. Forty patients developed diabetes during follow-up, 9 (5.9%) in the enalapril group and 31(22.4%) in the placebo group (P<0.0001). By multivariate analysis, enalapril remained the most powerful predictor for risk reduction of developing diabetes (hazard ratio, 0.22; 95% confidence intervals, 0.10 to 0.46; P<0.0001). Enalapril therefore significantly reduces the incidence of diabetes in patients with left ventricular dysfunction, especially those with impaired FPG.

### FIELD OF THE INVENTION

The present invention relates to the prevention of diabetes mellitus in a subject with chronic heart failure. Specifically, the present invention concerns the use of an angiotensin-converting enzyme (ACE) inhibitor, such as enalapril, to lessen the chances that a subject with left ventricular systolic dysfunction, whether symptomatic or not, will develop diabetes.

### BACKGROUND OF THE INVENTION

Diabetes mellitus is a common comorbidity in patients with heart failure; it is present at baseline in 20% to 25% of the subjects enrolled in large randomized clinical trials.¹⁻³ Furthermore, the presence of diabetes is an independent predictor of morbidity and mortality in these patients,⁴⁻⁵ almost doubling their incidence of death or hospitalization for cardiovascular reasons.⁵ Angiotensin-converting enzyme (ACE) inhibitors reduce mortality and the need for hospitalization and improve functional status in a wide array of heart failure patients (New York Heart Association class I to IV).^{2,6} In diabetic patients, ACE inhibitors prevent the development and progression of incipient or established nephropathy^{7,8} and delay the progression of diabetic retinopathy.⁹ Recently, the ACE inhibitor ramipril was demonstrated to reduce death, cardiovascular events (myocardial infarction and stroke), progression of diabetic nephropathy, and even the number of new cases of diabetes in high risk patients.^{10,11} However, there are no available data on the impact of long-term ACE inhibitor therapy on the incidence of diabetes in a cohort of patients with left ventricular dysfunction. Thus, to evaluate the effect of ACE inhibition on the development of diabetes in a heart failure population, a retrospective analysis of the Montreal Heart Institute patients who have been enrolled in the SOLVD trials (Studies of Left Ventricular Dysfunction) was conducted. While a specific objective of this study was to assess the impact of the ACE inhibitor enalapril on the development of diabetes in patients with left ventricular dysfunction, a related goal was to determine how ACE inhibition might contribute to prevent the onset of diabetes in patients with chronic heart failure.

### SUMMARY OF THE INVENTION

In accordance with the present invention, there is therefore provided a method of reducing the incidence of diabetes in a subject with chronic heart failure. Specifically, it has been found that the ACE inhibitor enalapril markedly reduces the risk of developing diabetes mellitus in patients with left ventricular systolic dysfunction, whether symptomatic or not. Since β-blockers appear to increase the risk of hyperglycemia and subsequent diabetes in heart failure patients, a combined therapy with an ACE inhibitor could also lessen the adverse effect of β-blockade.

In an embodiment of the present invention, the ACE inhibitor is chosen from the following group: enalapril (vasotec), captopril (capoten), lisinopril (prinivil, zestril), quinapril (accupril) and ramapril (altace). The ACE inhibitor is administered in a dosage of about 5-20 mg/day, as determined by the attending physician.

Like ACE inhibitors, angiotensin II receptor antagonists have an effect on the renin-angiotensin system. ACE inhibitors exert their effects earlier in the renin-angiotensin pathway than do angiotensin II receptor antagonists. Given the similarities in modes of action and overall effects caused by individual members of these two classes of compounds, it is believed that an angiotensin II receptor antagonist might be effectively used as an alternative (or substitute) to an ACE inhibitor in the prevention of diabetes in a subject with chronic heart failure. Suitable angiotensin II receptors include the following: losartan (cozaar), candesartan (atacand), irbesartan (avapro), telmisartan (micardis) and valsartan (diovan).

Subjects who are most likely to benefit from the present invention include individuals suffering from left ventricular systolic dysfunction (whether symptomatic or not) or hypertension, as well as individuals with other heart ailments who are predisposed to diabetes.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

FIG. 1: Kaplan-Meier curves for the time to occurrence of diabetes for the 291 patients in the enalapril (solid line) and placebo (dotted line) groups (P<0.0001).

FIG. 2: Kaplan-Meier curves for the time to occurence of diabetes in the subgroup of patients with impaired FPG at baseline in the enalapril (solid line) and placebo (dotted line) groups (P<0.0001).

### DESCRIPTION OF THE PREFERRED EMBODIMENTS

### EXPERIMENTAL

Study population

The patients from the Montreal Heart Institute who were randomized in the SOLVD trials were included in this study. SOLVD was a multicenter, double-blind, randomized, placebo-controlled trial that assessed the effect of the ACE inhibitor enalapril on survival in patients with left ventricular dysfunction (ejection fraction≦35%). The details of the trial have been described elsewhere.¹² Briefly, the prevention trial included 4228 patients with asymptomatic left ventricular dysfunction, and the treatment trial randomized 2569 patients with congestive heart failure from June 1986 to August 1991. Patients were randomized to enalapril (5 to 20 mg/day) or placebo. Exclusion criteria included age >80 years, unstable angina pectoris, myocardial infarction in the previous month, severe pulmonary disease, renal insufficiency (creatinine level>177 μmol/L [2 mg/dL]), and intolerance to ACE inhibitor or current ACE inhibitor use. Follow-up visits were scheduled 2 and 6 weeks after randomization and every 4 months until the end of the study, for a mean follow-up of 3.4 and 3.1 years for the treatment and prevention trials, respectively.

Data Collection and Definitions

Baseline characteristics, past medical history, and medication profiles at the time of enrolment into the SOLVD trials were obtained from the SOLVD databases. Fasting plasma glucose (FPG) was not collected for research purposes in the SOLVD trials. However, follow-up of patients in SOLVD involved regular blood samples, including FPG, at almost every research visit. Accordingly, the medical file of each patient was reviewed, and FPG results were collected. Chart reviewers were blinded to treatment allocation.

A diagnosis of new onset diabetes during the follow-up period was defined according to the American Diabetes Association criteria¹³ as a FPG≧126 mg/dL (7.0 mmol/L) at 2 different visits. For the purpose of the present study, the visits in which FPG≧126 occurred during infection, trauma, or acute myocardial infarction were not included.

Participants with diabetes at baseline (history of diabetes or FPG≧126 mg/dL at screening visit) were excluded. The study population was further divided among patients with impaired FPG at baseline (110 mg/dL[6.1 mmol/L]≦FPG<126mg/dL [7.0 mmol/L]) and those with normal FPG at baseline (FPG<110 mg/dL).

Statistical Analysis

The baseline characteristic of the 2 groups were compared using Student's t test for continuous variables and the X² test for categorical variables. Incidence of diabetes in the 2 groups was compared with the X² test. Time to occurrence of diabetes during the follow-up was analyzed with Kaplan-Meier curves and compared with the log-rank test. To analyze the effect of the treatment (enalapril) on development of diabetes, a Cox regression analysis was used to take into account the effect of potential confounding baseline variables (age, sex, current smoking, history of hypertension, and weight) and time-dependent variables (systolic blood pressure; diastolic blood pressure; and use of β-blockers, diuretics, calcium-channel blockers, antiplatelet agents, or antiarrhythmics). Cox proportional-hazard models were performed for each variable, with treatment (enalapril) forced in all models. Variables with a P≦0.2 were included in a multivariate Cox proportional hazard model. For time-dependent variables, the last value before the occurrence of diabetes was taken; if the patient did not develop diabetes, the value at the last visit was taken. Subgroup analyses were conducted with the X² test. In the particular subgroup of patients with impaired FPG at baseline, Kaplan-Meier curves were performed. Preliminary assumptions were verified before all analyses. P<0.05 was considered significant. All analyses were performed using SAS version 8.2 (SAS Institute, Inc).

Results

Study Population

Among the 391 patients from the Montreal Heart Institute who were randomized in SOLVD (prevention and treatment arms), 80 had a diagnosis of diabetes at randomization and 20 had insufficient data regarding FPG. The remaining 291 patients constituted the study population: 198 were in the prevention arm and 93 were in the treatment arm. Of these 291 patients, 153 were randomized to enalapril and 138 to placebo. The mean follow-up of the patients was 2.9±1.0 years (range, 0.2 to 4.8 years). FPG samples were collected at almost every research visit (mean of 7.9±3.5 samples per patient) during the study follow-up.

TABLE 1
Baseline Patient Characteristic in the 2 Groups

| | Enalapril | Placebo | |
|---|---|---|---|
| Characteristics | (n = 153) | (n = 138) | P |
| Age, y | 56.1 ± 10.1 | 56.8 ± 10.0 | 0.581 |
| Male sex, % | 90.9 | 93.5 | 0.406 |
| Weight, kg | 74.9 ± 10.7 | 76.0 ± 12.0 | 0.510 |
| Systolic blood pressure, mm Hg | 127.4 ± 17.3 | 128.2 ± 16.7 | 0.708 |
| Diastolic blood pressure, mm Hg | 77.8 ± 8.6 | 79.7 ± 8.4 | 0.057 |
| Heart rate, bpm | 74.5 ± 10.2 | 75.0 ± 11.0 | 0.741 |
| NYHA class, % | | | 0.778 |
| I | 28.8 | 31.2 | |
| II | 66.0 | 62.3 | |
| III | 5.2 | 6.5 | |
| Current smoking, % | 43.9 | 39.2 | 0.440 |
| Past history, % | | | |
| Hypertension | 19.0 | 17.4 | 0.730 |
| Cerebrovascular accident | 7.2 | 9.4 | 0.489 |
| Previous MI | 91.5 | 85.5 | 0.107 |
| Primary cause of LV | | | 0.388 |
| dysfunction, % | | | |
| Ischemic | 92.2 | 91.3 | |
| Other | 7.8 | 8.7 | |
| LV ejection fraction, % | 26.0 ± 7.0 | 26.5 ± 6.5 | 0.555 |
| Serum creatinine, mg/dL | 1.0 ± 0.2 | 1.0 ± 0.2 | 0.727 |
| Drug therapy, % | | | |
| Diuretics | 45.8 | 42.8 | 0.607 |
| β-Blockers | 18.3 | 21.7 | 0.463 |
| Calcium-channel blockers | 37.9 | 45.9 | 0.266 |
| Antiplatelet agents | 43.8 | 37.7 | 0.289 |
| Antiarrhythmic agents | 9.8 | 12.3 | 0.493 |

Data are presented as the mean ± SD or percentages of patients.
NYHA indicates New York Heart Association;
MI, myocardial infarction; and
LV, left ventricular.

Baseline Characteristics

The baseline characteristics of the 291 patients were well balanced between the 2 groups and are provided in Table 1. Most patients were men with NYHA class II symptoms and severe systolic dysfunction (mean ejection fraction of 26%) of ischemic cause. Approximately 20% of patients were receiving a β-blocker and 45% were treated with diuretics.

Development of Diabetes

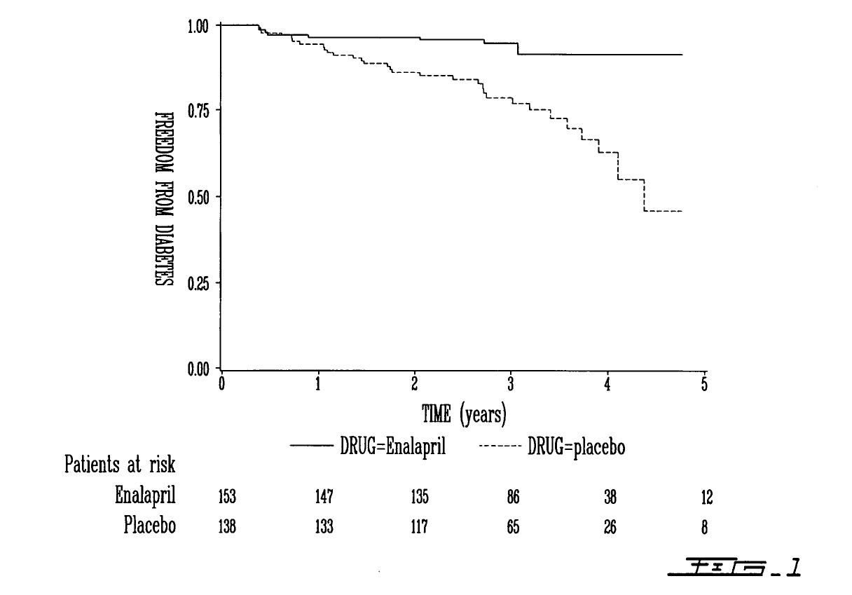

Forty patients met the criteria for new-onset diabetes during the follow-up period, 9 (5.9%) in the enalapril group and 31 (22.4%) in the placebo group (relative risk [RR], 0.26; P<0.0001). This represents an absolute risk reduction of 16.5%. During the follow-up, the probability of remaining free from diabetes was significantly higher with enalapril than with placebo (P<0.0001; FIG. 1). By multivariate analysis using a Cox regression model (Table 2), enalapril treatment remained the most powerful variable associated with decreased risk of developing diabetes (hazard ratio, 0.22; 95% confidence intervals, 0.10 to 0.46; P<0.0001). Age was the only other variable remaining in the model that was significantly related to the development of diabetes (hazard ratio, 1.05; 95% confidence intervals, 1.01 to 1.08; P=0.005).

The effect of enalapril in a subgroup of patients known to be at high risk for diabetes, ie, those with impaired FPG at baseline, was also examined. Only 1 patient developed diabetes in the enalapril group compared with 12 patients in the placebo group, which represent an absolute risk reduction of 45% (Table 3).

TABLE 2
Univariate and Mutivariate Analyses of Risk Factors for the
Development of Diabetes

| Variables | P | Hazard Ratio |
|---|---|---|
| Univariate analysis | | |
| At baseline | | |
| Age | 0.005 | . . . |
| Sex | 0.740 | . . . |
| Current smoking | 0.724 | . . . |
| History of hypertension | 0.390 | . . . |
| Weight | 0.585 | . . . |
| Time-Dependent | | |
| SBP | 0.567 | . . . |
| DBP | 0.814 | . . . |
| ΔSBP | 0.786 | . . . |
| ΔDBP0 | 0.730 | . . . |
| Drug therapy | | |
| β-Blockers | 0.471 | . . . |
| Diuretics | 0.779 | . . . |
| Calcium-channels blockers | 0.777 | . . . |
| Antiplatelet agents | 0.851 | . . . |
| Antiarrhythmic | 0.354 | . . . |
| Multivariate analysis | | |
| Drug therapy: enalapril | <0.0001 | 0.22 |
| Age | 0.005 | 1.05 |

SBP indicates systolic blood pressure;
DBP, diastolic blood pressure;
ΔSBP, SBP at baseline minus SBP at the end of the study; and
ΔDBP, DBP at baseline minus DBP at the end of the study.

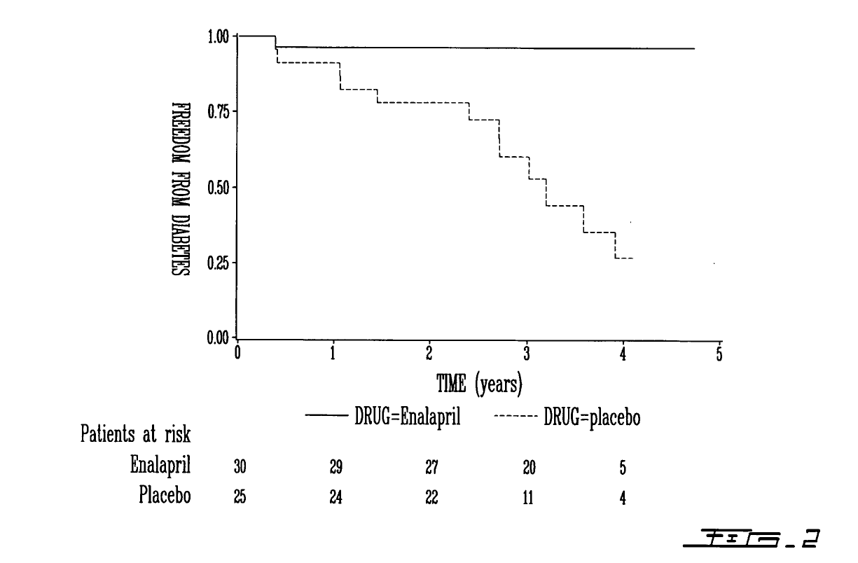

Kaplan-Meier curves for time to occurrence of diabetes in this subgroup of patients are shown in FIG. 2. The analysis was further stratified according to baseline functional status by analyzing the effect of enalapril in the 2 arms of the trial (prevention and treatment). The beneficial effect of enalapril on the development of diabetes was significant, regardless of functional status at baseline (Table 3).

TABLE 3
Effect of Enalapril on the Number of New Cases of Diabetes
According to Baseline FPG and Trial Arm

| | Enalapril, | Placebo, | Absolute Risk | |
|---|---|---|---|---|
| | n (%) | n (%) | Reductlon, % | P |
| Baseline FPG | | | | |
| IFG (n = 55) | 1(3.3) | 12(48.0) | 44.7 | 0.0001 |
| NFG (n = 236) | 8(6.6) | 19(17.3) | 10.7 | 0.011 |
| Arm of the trial | | | | |
| Prevention (n = 198) | 6(6.0) | 19(19.4) | 13.4 | 0.004 |
| Treatment (n = 93) | 3(5.7) | 12(30.0) | 24.3 | 0.001 |
| Total population | 9(5.9) | 31(22.4) | 16.5 | <0.0001 |
| (n = 291) | | | | |

IFG indicates impaired FPG;
NFG, normal FPG.

Discussion

The above results reveal that in nondiabetic patients with left ventricular systolic dysfunction, the ACE inhibitor enalapril markedly reduced the risk of developing diabetes. Although retrospective in approach, the present study deserves attention for several reasons. First, the baseline characteristics, including medications, were well balanced between the 2 groups. Second, even if these results are derived from a study that was published >10 years ago, the findings are still very relevant to current clinical practice. This is particularly true because the standard heart failure treatment now includes β-blockers, a class of drug that lowers mortality when combined with ACE inhibitors but seems to increase the risk of diabetes¹⁴ (perhaps except for carvedilol¹⁵, which has been shown to have a favorable effect on insulin sensitivity compared with metoprolol). Furthermore, the prognosis of heart failure is worse when it is associates with diabetes.⁴

The present study extends the beneficial effects of ACE inhibitors on the prevention of diabetes to all patients with left ventricular systolic dysfunction, whether symptomatic or not. Patients with impaired FPG were particularly likely to benefit. Other randomized trials, including CAPP (CAptopril Prevention Project) have demonstrated a reduction in the relative risk of developing diabetes (RR=0.86; P=0.039) when an hypertensive population was treated with captopril compared with a β-blocker or a diuretic.¹⁶ Similar results were obtained in the LIFE (Losartan Intervention For Endpoint) trial,¹⁷ in which losartan was compared with atenolol in patients with hypertension (6% developed diabetes in the losartan group versus 8% with atenolol; RR=0.75; P=0.001). Of note, 2 different levels of serum glucose were used to diagnose diabetes as the criteria evolved during that study.

Also, a nonsignificant 20% relative reduction in the incidence of diabetes was found in the recently presented Study on Cognition and Prognosis in the Elderly study when candesartan was compared with placebo (L. Hansson, MD, University of Uppsala, Sweden, unpublished data, 2002); however, β-blockers were used more frequently in the placebo group than in the candesartan group. From these studies, it cannot be concluded whether these findings were the result of a beneficial effect of captopril, losartan, or candesartan or a detrimental effect of β-blockers on diabetes. The Heart Outcomes Prevention Evaluation (HOPE) study has demonstrated a reduction in the number of new cases of diabetes with ramipril.¹¹ Although the development of diabetes was not a predetermined end point in HOPE, Yusuf et al.¹¹ have shown, with a treatment period of 4.5 years, that ramipril reduced the relative risk of developing diabetes by 34% (3.6% in ramipril group versus 5.4% in placebo group; RR=0.66; P<0.001) in a cohort of high-risk patients with no evidence of left ventricular dysfunction. The incidence of diabetes observed in the placebo group of the present study (22.4%) is much higher than in that of the HOPE study (5.4%).

This can be explained by many factors, including the fact that a strict biochemical definition of diabetes was used (i.e., with FPG level), whereas the diagnosis in HOPE was based on the patients' self-report of newly diagnosed diabetes, thus resulting in an underestimation of the true incidence in that trial. Second, the severity of the underlying disease (established left ventricular dysfunction in SOLVD) may also contribute to the difference in incidence in these trials. Indeed, the neurohormonal activation encountered in heart failure can both increase peripheral insulin resistance and decrease insulin secretion, thus leading to impaired glucose handling,¹⁸ which favors the development of diabetes.¹⁹ The difference in the incidence of diabetes between both trials is even more striking considering that the follow-up was much longer in HOPE than in the present study (4.5 years versus 2.9 years).

The mechanisms by which ACE inhibition exerts its protective effect against diabetes are not completely understood. ACE inhibitors not only block the conversion of angiotension I to angiotensin II but also increase bradykinin levels through inhibition of kininase II-mediated degradation.²⁰⁻²¹ In hypertensive rats, Tomiyama and coworkers²² have shown improved insulin sensitivity with enalapril through an increase in endogenous kinins. The higher kinin levels lead to increased production of prostaglandins (PGE₁ and PGE₂) and nitric oxide, which improve muscle sensitivity to insulin²³⁻²⁵ and exercise-induced glucose metabolism,²⁶ resulting in enhanced insulin-mediated glucose uptake. Furthermore, the peripheral vasodilatory actions of ACE inhibitors (through diverse mechanisms, including prostaglandin and nitric oxide) lead to an improvement in skeletal muscle blood flow, the primary target for insulin action and an important determinant of glucose uptake.²⁷ Clinical evidence supporting this effect has been provided by Morel and coworkers,²⁸ who have shown improved insulin sensitivity when enalapril was given for 12 weeks to 14 obese, hypertensive, and dyslipidemic patients. A similar effect has also been reported with captopril.²⁹ Finally, ACE inhibitors inhibit the vasoconstrictive effect of angiotensin II in the pancreas and increase islet blood flow,³⁰ which could improve insulin release by β-cells. The present experimental and clinical studies all support these findings and suggest that ACE inhibition increases insulin sensitivity, skeletal muscle glucose transport, and pancreatic blood flow, which probably all contribute to the prevention of diabetes mellitus.

Clinical Implications

Diabetes mellitus is a major risk factor for cardiovascular events, increasing morbidity and mortality in heart failure patients. The lower incidence of diabetes found in heart failure patients treated with the ACE inhibitor enalapril should lead to improved long-term cardiovascular prognosis in this population. Because β-blockers seem to increase the risk of hyperglycemia and subsequent diabetes, combined therapy with an ACE inhibitor could attenuate this adverse effect of β-blockade. With an absolute risk reduction of 16.5% with enalapril in the present study, it is necessary to treat 6 patients with left ventricular dysfunction for 2.9 years to prevent one new case of diabetes.

Limitations

The present analysis was not a prespecified end point of the SOLVD trials, and FPG levels were not measured as an integral part of the trials. Nevertheless, FPG samples were measured serially for clinical purposes, and their results were carefully reviewed. The present results reflect the true incidence of diabetes in patients with left ventricular dysfunction, using strict and modern diagnosis criteria of diabetes.

Conclusion

The ACE inhibitor enalapril markedly reduces the risk of developing diabetes mellitus in patients with left ventricular dysfunction. This beneficial effect is even more striking in patients with impaired FPG.

Although the present invention has been described hereinabove by way of preferred embodiments thereof, it can be modified without departing from the spirit, scope and nature of the subject invention, as defined in the appended claims.

List of References

1. The CONSENSUS trial study group. Effect of enalapril on mortality in severe congestive heart failure. N Engl J Med. 1987; 316:1429-1435.

2. The SOLVD Investigators. Effect of enalapril on mortality and the development of heart failure in asymptomatic patients with reduced left ventricular ejection fractions. N Engl J Med. 1992: 327: 685-691.

3. Packer M, Poole-Wilson P A, Armstrong P W, et al. Comparative effects of low doses and high doses of the angiotensin converting enzyme inhibitor lisinopril on morbidity and mortality in chronic heart failure: ATLAS study group. Circulation. 1999; 23: 2312-2318.

4. Shindler D M, Kostis J B, Yusuf S, et al. Diabetes mellitus, a predictor of morbidity and mortality in the Studies of Left Ventricular Dysfunction (SOLVD) trials and registry. Am J Cardiol. 1996; 77: 1017-1020.

5. Bourassa M G, Gurné O, Bangdiwala S I, et al. Natural history and patterns of current practice in heart failure. J Am Coll Cardiol. 1993; 22(suppl A): 14A-19A.

6. The SOLVD Investigators. Effect of enalapril on survival in patients with reduced left ventricular ejection fractions and congestive heart failure. N Engl J Med, 1991; 325: 293-302.

7. Lewis E J, Hunsicker L G, Bain R P, et al. The effect of angiotensin converting-enzyme inhibition on diabetic nephropathy. N Engl J Med. 1993; 329: 1456-1462.

8. The ACE Inhibitors in Diabetic Nephropathy Trialist Group. Should all patients with type 1 diabetes mellitus and microalbuminuria receive angiotensin-converting enzyme inhibitors? A meta-analysis of individual patient data. Ann Intern Med. 2001;134: 370-379.

9 Parving H H, Larsen M, Hommel E, et al. Effect of antihypertensive treatment on blood-retinal barrier permeability to fluorescein in hypertensive type-1 diabetic patients with background retinopathy Diabetologia 1989; 32: 440-444.

10. The Heart Outcomes Prevention Evaluation Study Investigators. Effects of an angiotensin-converting-enzyme inhibitor, ramipril, on cardiovascular events in high risk patients. N Engl J Med. 2000; 342: 145-153.

11. Yusuf S, Gerstein H, Hoogwerf B, et al. Ramipril and the development of diabetes. JAMA. 2001;286: 1882-1485.

12. The SOLVD Investigators. Studies of Left Ventricular Dysfunction (SOLVD): rationale, design and methods: two trials that evaluate the effect of enalapril in patients with reduced ejection fraction. Am J Cardiol. 1990;66:315-322.

13. American Diabetes Association. Report of the expert committee on the diagnosis and classification of diabetes mellitus. Diabetes Care. 1997;20:1183-201.

14. Gress T W, Nieto F J, Shahar E, et al. Hypertension and antihypertensive therapy as risk factors for type 2 diabetes mellitus. N Engl J Med. 2000;342:905-912.

15. Jacob S, Rett K, Wickimayr M, et al. Differential effect of chronic treatment with two beta-blocking agents on insulin sensitivity: the carvedilol-metoprolol study. J Hypertens. 1996; 14:489494.

16. Hansson L, Lindholm L H, Niskanen L, et al. Effects of angiotensin converting-enzyme inhibition compared with conventional therapy on cardiovascular morbidity and mortality in hypertension: the Captopril Prevention Project (CAPPP) randomised trial. Lancet. 1999;353:611-616.

17. Lindholm L H, Ibsen H, Borch-Johnsen K, et al. Risk of new-onset diabetes in the losartan intervention for endpoint reduction in hypertension study. J Hypertens. 2002;20:1879-1886.

18. Paolisso G, De Riu S, Marrazzo G, et al. Insulin resistance and hyperinsulinemia in patients with chronic congestive heart failure. Metabolism. 1991 ;40:972-977.

19. Sacks D B, Mc Donald J M. The pathogenesis of type II diabetes mellitus: a polygenic disease. Am J Clin Pathol. 1996;105:149-156.

20. Uehara M, Kishikawa H, Isami S, et al. Effect on insulin sensitivity of angiotensin converting enzyme inhibitors with or without a sulphydryl group: bradykinin may improve insulin resistance in dogs and humans. Diabetologia. 1994;37:300-307.

21. Yang H Y, Erdös E G, Levin Y. A dipeptidyl cardoxypeptidase that converts angiotensin I and inactivates bradykinin. Biochim Biophys Acta. 1970;214:374-376.

22. Tomiyama H, Kushiro T, Abeta H, et al. Kinins contribute to the improvement of insulin sensitivity during treatment with angiotensin converting enzyme inhibitor. Hypertension. 1994;23 :450-455.

23. Leighton B, Budohoski L, Lozeman F J, et al. The effect of prostaglandins E₁, E₂ and F_{2α}and indomethacin on the sensitivity of glycolysis and glycogen synthesis to insulin in stripped soleus muscles of the rat. Biochem J. 1985;27:337-340.

24. Fryer L G D, Hajduch E, Rencurel F, et al. Activation of glucose transport by AMP-activated protein kinase via stimulation of nitric oxide synthase. Diabetes. 2000;49: 1978-1985.

25. Henriksen E J, Jacob S, Kinnick T R, et al. ACE inhibition and glucose transport in insulin-resistant muscle: roles of bradykinin and nitric oxide. Am J Physiol. 1999;277:R332-R336.

26. Balon T W, Nadler J L. Evidence that nitric oxide increases glucose transport in skeletal muscle. J Appl Physiol. 1997;82:359-363.

27. Shultz T A, Lewis S B, Westbie D K, et al. Glucose delivery: a modulator of glucose uptake in contracting skeletal muscle. Am J Physiol. 1977;2:E514-E518.

28. Morel Y, Gadient A, Keller U, et al. Insulin sensitivity in obese hypertensive dyslipidemic patients treated with enalapril or atenolol. J Cardiovascular Pharmacol. 1995;26:306-311.

29. Pollare T, Lithell H, Beme C. A comparison of the effects of hydrochlorothiazide and captopril on glucose and lipid metabolism in patients with hypertension. N Engl J Med. 1989:321:868-873.

30. Carlsson P O, Beme C, Jansson L. Angiotensin II and the endocrine pancreas: effects on islet blood flow and insulin secretion in rats. Diabetologia. 1998;41:127-133.

What is claimed is:

1. A method for preventing the incidence of diabetes mellitus in a subject with chronic heart failure comprising the administration of a therapeutically effective amount of an ACE inhibitor.

2. A method as defined in claim 1, wherein said subject has impaired fasting plasma glucose (FMG).

3. A method as defined in claim 1, wherein said chronic heart failure is a result of symptomatic or asymptomatic left ventricular systolic dysfunction.

4. A method as defined in claim 3, wherein said chronic heart failure is a result of symptomatic left ventricular systolic dysfunction.

5. A method as defined in claim 1, wherein said ACE inhibitor is selected from the group consisting of: enalapril (vasotec), captopril (capoten), lisinopril (prinivil, zestril), quinapril (accupril) and ramapril (altace).

6. A method as defined in claim 5, wherein said ACE inhibitor is administered in a dosage of about 5-20 mg/day.

7. A method as defined in claim 6, wherein said ACE inhibitor is enalapril.

8. A method for preventing the incidence of diabetes mellitus in a subject with chronic heart failure that is being treated with a β-blocker comprising the administration of a therapeutically effective amount of an ACE inhibitor.

9. A method as defined in claim 8, wherein said subject has impaired fasting plasma glucose (FMG).

10. A method as defined in claim 8, wherein said chronic heart failure is a result of symptomatic or asymptomatic left ventricular systolic dysfunction.

11. A method as defined in claim 10, wherein said chronic heart failure is a result of symptomatic left ventricular systolic dysfunction.

12. A method as defined in claim 8, wherein said ACE inhibitor is selected from the group consisting of: enalapril (vasotec), captopril (capoten), lisinopril (prinivil, zestril), quinapril (accupril) and ramapril (altace).

13. A method as defined in claim 12, wherein said ACE inhibitor is administered in a dosage of about 5-20 mg/day.

14. A method as defined in claim 13, wherein said ACE inhibitor is enalapril.

15. A method as described in claim 14, wherein said β-blocker is metoprolol.

16. A method for preventing the incidence of diabetes mellitus in a subject with chronic heart failure comprising the administration of a therapeutically effective amount of angiotensin II receptor antagonist.

17. A method as defined in claim 16, wherein said subject has impaired fasting plasma glucose (FMG).

18. A method as defined in claim 16, wherein said chronic heart failure is a result of symptomatic or asymptomatic left ventricular systolic dysfunction.

19. A method as defined in claim 18, wherein said chronic heart failure is a result of symptomatic left ventricular systolic dysfunction.

20. A method as defined in claim 16, wherein said angiotensin II receptor antagonist is selected from the group consisting of: losartan (cozaar), candesartan (atacand), irbesartan (avapro), telmisartan (micardis) and valsartan (diovan).

21. A method as defined in claim 20, wherein said angiotensin II receptor antagonist is administered in a dosage of about 5-20 mg/day.

22. A method as defined in claim 21, wherein said ACE inhibitor is losartan.
